(19) United States
(12) Patent Application Publication
(10) Pub. No.: US 2015/0283070 A1
(43) Pub. Date: Oct. 8, 2015

(54) Nicotine Formulations and Methods of Making the Same

(71) Applicant: Sansa Corporation (Barbados) Inc., Worthing (BB)

(72) Inventors: Alex Stenzler, Long Beach, CA (US); Arthur Slutsky, Ontario (CA); Noe Zamel, Ontario (CA)

(21) Appl. No.: 14/681,859

(22) Filed: Apr. 8, 2015

Related U.S. Application Data
(60) Provisional application No. 61/976,712, filed on Apr. 8, 2014.

(51) Int. Cl. A61K 9/00; A61K 9/16; A61K 31/045; A61K 45/06; A24F 47/00; A61K 31/465

(52) U.S. Cl. CPC A61K 9/0075; A24F 47/002; A61K 31/465; A61K 31/045; A61K 45/06; A61K 9/1694; A61K 9/1623

(57) ABSTRACT

A dry powder nicotine formulation suitable for inhalation is described. The formulation includes nicotine particles, wherein the nicotine particles are substantially in the range of about 1-10 micron in size, preferably 2-5 micron in size. The formulation may also include a cough suppressant component having particles in the 5-100 micron size range. The formulation may also include a cough suppressant component having particles in the 10-200 micron size range. The formulation may also include a flavor component having particles in the 10-1000 micron size range.

### CROSS-REFERENCE TO RELATED APPLICATIONS

This application claims priority to U.S. patent application Ser. No. 61/976,712, filed Apr. 8, 2014, the entire contents of which are incorporated herein by reference.

### BACKGROUND OF THE INVENTION

It is believed that cigarette smoke contains approximately 4000 chemical compounds and has a range of particle sizes from less than 0.1 micron to approximately 0.5 micron. During inhalation, it is known that most particles larger than 10-12 micron in size typically cannot make the turn in the oral cavity to enter the lower respiratory tract and instead impact the back of the throat. While particles less than 5 micron in size are generally considered respirable and can thus enter the lower respiratory tract, the majority of particles less than 1 micron in size do not settle in the alveoli, and are thus expelled during subsequent exhalation. Consequently, exhaled particles of this size range (less than about 1 micron) are commonly characterized as “second hand smoke.”

The state of the art in the development of products designed to replace traditional cigarettes, is to replicate or match the particles found in cigarettes. For example, such replacement technologies include e-cigarettes that produce nicotine vapor, ultrasonically produced nicotine aerosol droplets or nicotine oral sprays. These replacement cigarette technologies typically produce particles that are less than 0.5 micron in size, and very large particles that are greater than 10-12 micron in size. However, each of these technologies suffer from the same result in that less than half of the inhaled nicotine and associated compounds remain in the lungs and the balance is exhaled into the environment. Unfortunately, this means that the public must still contend with the same problem of users of these technologies producing what is effectively second hand smoke, and accordingly these technologies are increasingly being banned in selected public spaces.

Thus, there is a need in the art for a nicotine-based formulation that uniquely targets the smaller airways of the lungs while reducing or eliminating exhalable nicotine by a subject. The present invention satisfies this need.

### SUMMARY OF THE INVENTION

The present invention relates to a dry powder nicotine formulation suitable for inhalation. The formulation includes nicotine particles that are substantially between about 1-10 micron in size. In one embodiment, the nicotine particles are substantially between about 2-5 micron in size. In another embodiment, less than about 10% of the nicotine particles are less than about 1 micron in size. In another embodiment, less than about 10% of the nicotine particles are less than about 2 micron in size. In another embodiment, at least about 90% of the nicotine particles are less than about 10 micron in size. In another embodiment, at least about 90% of the nicotine particles are less than about 5 micron in size. In another embodiment, less than about 10% of the nicotine particles are less than about 1 micron in size and wherein at least about 90% of the nicotine particles are less than about 10 micron in size. In another embodiment, less than about 10% of the nicotine particles are less than about 2 micron in size and wherein at least about 90% of the nicotine particles are less than about 5 micron in size.

The present invention also relates to a dry powder nicotine formulation suitable for inhalation that includes a nicotine based component having particles substantially between about 1-10 micron in size, and a cough suppressant component having particles substantially between about 5-10 micron in size. In one embodiment, the cough suppressant component comprises menthol. In another embodiment, the nicotine based component particles are substantially between about 2-5 micron in size and the cough suppressant component particles are substantially between about 5-8 micron in size. In another embodiment, the formulation further includes a cough suppressant component having particles substantially between about 10-200 micron in size. In another embodiment, the cough suppressant component having particles substantially between about 10-200 micron in size comprises menthol. In another embodiment, the formulation further includes a flavor component having particles substantially between about 10-1000 micron in size. In another embodiment, the flavor component comprises menthol.

The present invention also relates to a method of producing a dry powder nicotine formulation suitable for inhalation. The method includes the steps of preparing a flowable mixture comprising nicotine and a sugar in a liquid carrier, and spray drying the flowable mixture to produce dry powder particles comprising nicotine and sugar that are substantially between about 1 micron in size and about 10 micron in size. In one embodiment, the sugar is lactose. In another embodiment, the lactose is non-spheronized. In another embodiment, the liquid carrier is water. In another embodiment, the liquid carrier comprises water and alcohol.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a flowchart depicting an exemplary method of manufacturing a formulation of the present invention.

FIG. 2 is a flowchart depicting another exemplary method of manufacturing a formulation of the present invention.

FIG. 3 is a flowchart depicting yet another exemplary method of manufacturing a formulation of the present invention.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless stated otherwise, the described size or size range of a particle should be considered as the mass median aerodynamic diameter (MMAD) of the particle or set of particles. Such values are based on the distribution of the aerodynamic particle diameters defined as the diameter of a sphere with a density of 1 gm/cm³ that has the same aerodynamic behavior as the particle which is being characterized. Because the particles described herein may be in a variety of densities and shapes, the size of the particles is expressed as the MMAD and not the actual diameter of the particles.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to dry powder formulations of nicotine, and optionally other selected materials, wherein the nicotine component and optional additional components fall within controlled particle size ranges. For example, in one embodiment, the formulation includes nicotine particles (also referred to herein as the nicotine-based component) sized substantially between 1-10 microns, based on the MMAD of the particles. In yet another embodiment, the formulation includes nicotine particles sized substantially between 1-7 microns. In another embodiment, the formulation includes nicotine particles sized substantially between 2-5 microns. In yet another embodiment, the formulation includes nicotine particles sized substantially between 2-3 microns. By selectively limiting or excluding nicotine particles below about 1 micron in size, or below about 2 microns in size, the formulations of the present invention remove or at least reduce a subject's ability to exhale nicotine back into the environment, thereby effectively reducing or removing the production of the nicotine contained in second hand smoke. Further, by selectively limiting or excluding non-respirable nicotine particles, the formulations of the present invention reduces unwanted irritation caused by nicotine particles trapped in the larger airways, oro-pharynx, the glottis vocal cords, and other anatomic regions more proximal or closer to the mouth.

Accordingly, in some embodiments, the smallest particles within the nicotine particle size range are at least about 1 micron, at least about 1.1 micron, at least about 1.2 micron, at least about 1.3 micron, at least about 1.4 micron, at least about 1.5 micron, at least about 1.6 micron, at least about 1.7 micron, at least about 1.8 micron, at least about 1.9 micron, or at least about 2 micron. In some embodiments, the largest particles within the nicotine particle size range are no greater than about 10 micron, no greater than about 7 micron, no greater than about 6 micron, no greater than about 5 micron, no greater than about 4.5 micron, no greater than about 4 micron, no greater than about 3.5 micron, or no greater than about 3 micron. In certain embodiments, no more than about 10% of the nicotine particles are less than about 1 micron. In certain embodiments, no more than about 10% of the nicotine particles are less than about 2 micron. In other embodiments, at least 90% of the nicotine particles are less than about 10 micron. In other embodiments, at least 90% of the nicotine particles are less than about 7 micron. In other embodiments, at least 90% of the nicotine particles are less than about 5 micron. In one embodiment, no more than about 10% of the nicotine particles are less than 1 micron and at least 90% of the nicotine particles are less than about 10 micron. In one embodiment, no more than about 10% of the nicotine particles are less than about 1 micron and at least 90% of the nicotine particles are less than about 7 micron. In one embodiment, no more than about 10% of the nicotine particles are less than about 2 micron and at least 90% of the nicotine particles are less than about 5 micron. In one embodiment, no more than about 10% of the nicotine particles are less than about 2 micron and at least 90% of the nicotine particles are less than about 3 micron.

In another example, the formulation of the present invention may optionally include a cough suppressant component having particles sized substantially between 5 and 10 microns. In one embodiment, the cough suppressant component is menthol. In one embodiment, the cough suppressant component is a sugar. In one such embodiment, the sugar is lactose. In another embodiment, the cough suppressant component may include benzocaine. It should be appreciated that the cough suppressant component can include any compound approved for suppressing cough. By selectively including menthol particles between 5-10 microns, these non-respirable menthol particles can reduce cough by soothing irritation in the subject's upper airways. Accordingly, in some embodiments, the smallest particles within the cough suppressant component particle size range are at least about 5 micron, at least about 6 micron, at least about 7 micron, or at least about 8 micron. In some embodiments, the largest particles within the cough suppressant component particle size range are no greater than about 10 micron, no greater than about 9 micron, no greater than about 8 micron, or no greater than about 7 micron. In certain embodiments, no more than about 10% of the cough suppressant particles are less than about 5 micron. In other embodiments, at least 90% of the cough suppressant particles are less than about 10 micron. In other embodiments, at least 90% of the cough suppressant particles are less than about 8 micron. In one embodiment, no more than about 10% of the cough suppressant particles are less than 4 micron and at least 90% of the cough suppressant particles are less than about 10 micron. In one embodiment, no more than about 10% of the cough suppressant particles are less than about 5 micron and at least 90% of the cough suppressant particles are less than about 8 micron. Although in the preferred embodiment the cough suppressant component is composed of particles substantially in the range of 5-10 micron, the cough suppressant component can comprise particles in a broader range. In one embodiment, the cough suppressant component can comprise particles in the range of 5-25 micron. In another embodiment, the cough suppressant component comprises particles substantially in the range of 5-50 micron. In yet another embodiment, the cough suppressant component comprises particles substantially in the range of 5-100 micron.

In another example, the formulation of the present invention may optionally include a flavor component having particles sized substantially between 10-1000 microns. In one embodiment, the flavor component is composed of particles substantially in the range of 10-200 micron. In a preferred embodiment, the flavor component is composed of particles substantially in the range of 10-100 micron. The flavor component utilizes such embedded larger particles that may impact the subject in the oral cavity to produce a desired flavor. Further, by limiting such flavor component particles to larger than 10 microns in size, these particles are limited in their ability to enter into the subject's lungs. Accordingly, in some embodiments, the smallest particles within the flavoring component particle size range are at least about 10 micron, at least about 12 micron, at least about 20 micron, at least about 30 micron, or at least about 50 micron. In some embodiments, the largest particles within the flavoring component particle size range are no greater than about 1000 micron, no greater than about 500 micron, no greater than about 200 micron, no greater than about 150 micron, no greater than about 120 micron, no greater than about 100 micron, no greater than about 90 micron, or no greater than about 80 micron. In certain embodiments, no more than about 10% of the flavor component particles are less than about 10 micron. In certain embodiments, no more than about 10% of the flavor component particles are less than about 20 micron.

In other embodiments, at least 90% of the flavor component particles are less than about 1000 micron. In other embodiments, at least 90% of the flavor component particles are less than about 500 micron. In other embodiments, at least 90% of the flavor component particles are less than about 200 micron. In other embodiments, at least 90% of the flavor component particles are less than about 150 micron. In other embodiments, at least 90% of the flavor component particles are less than about 100 micron. In one embodiment, no more than about 10% of the flavor component particles are less than 10 micron and at least 90% of the flavor component particles are less than about 1000 micron. In one embodiment, no more than about 10% of the flavor component particles are less than 10 micron and at least 90% of the flavor component particles are less than about 200 micron. In one embodiment, no more than about 10% of the flavor component particles are less than about 10 micron and at least 90% of the flavor component particles are less than about 100 micron. In one embodiment, the flavor component is menthol. In other embodiments, the flavor component may include tobacco, fruit flavors, or food-grade flavorings, for example the types of flavorings typically used in candy or baking It should be appreciated that the flavor compound may be any flavoring compound known in the art, preferably a regulatory-approved flavoring compound.

In another example, the formulation of the present invention may optionally include a cough suppressant component having particles sized substantially between 10-200 microns. This cough suppressant component can be added to the formulation instead of, or in addition to, the cough suppressant component in the range of 5-10 previously discussed. Accordingly, the formulation of the present invention can comprise two cough suppressant components, wherein each cough suppressant component has a substantially different particle size distribution. The 10-200 micron cough suppressant component may reduce a cough caused by irritation of the oro-pharynx, the glottis vocal cords, and other anatomic regions more proximal or closer to the mouth that contain receptors that can trigger cough or trigger other unwanted sensations. As contemplated herein, these larger particles are substantially prohibited from entering the sub-glottic airways. Accordingly, in some embodiments, the smallest particles within the cough suppressant component particle size range are at least about 10 micron, at least about 12 micron, at least about 20 micron, at least about 30 micron, or at least about 50 micron.

In some embodiments, the largest particles within the cough suppressant component particle size range are no greater than about 200 micron, no greater than about 150 micron, no greater than about 120 micron, no greater than about 100 micron, no greater than about 90 micron, or no greater than about 80 micron. In certain embodiments, no more than about 10% of the cough suppressant component particles are less than about 10 micron. In certain embodiments, no more than about 10% of the cough suppressant component particles are less than about 20 micron. In other embodiments, at least 90% of the cough suppressant component particles are less than about 200 micron. In other embodiments, at least 90% of the cough suppressant component particles are less than about 150 micron. In other embodiments, at least 90% of the cough suppressant component particles are less than about 100 micron.

In one embodiment, no more than about 10% of the cough suppressant component particles are less than 10 micron and at least 90% of the cough suppressant component particles are less than about 200 micron. In one embodiment, no more than about 10% of the cough suppressant component particles are less than about 12 micron and at least 90% of the cough suppressant component particles are less than about 100 micron. In one embodiment, the cough suppressant component includes menthol particles between 10-200 microns in size, which may provide a soothing effect in areas of particle impact. In another embodiment, the cough suppressant component having particles between 10-200 microns in size may include benzocaine. It should be appreciated that the cough suppressant component having particles between 10-200 microns in size can include any compound approved for suppressing cough. In another example, the addition of at least one component in the formulation of the present invention other than the nicotine component may act to dilute the nicotine containing particles and decrease cough caused by nicotine irritating the oro-pharynx, vocal cords, and other anatomic regions proximal to the trachea.

Accordingly, the formulations and methods of the present invention represent a novel product and approach to dry powder nicotine-based formulations. Unlike existing technologies which do not separate or segregate material components according to size, composition or any other parameter, the present invention selectively limits particular material components of the formulation to specific and controlled particle size ranges, thereby providing a unique and superior product that delivers respirable nicotine to the alveoli and small airways while reducing or eliminating exhaled nicotine; optionally delivers a non-respirable cough suppressant to the larger airways and/or the oro-pharynx; and optionally delivers non-respirable flavor particles to the oral cavity.

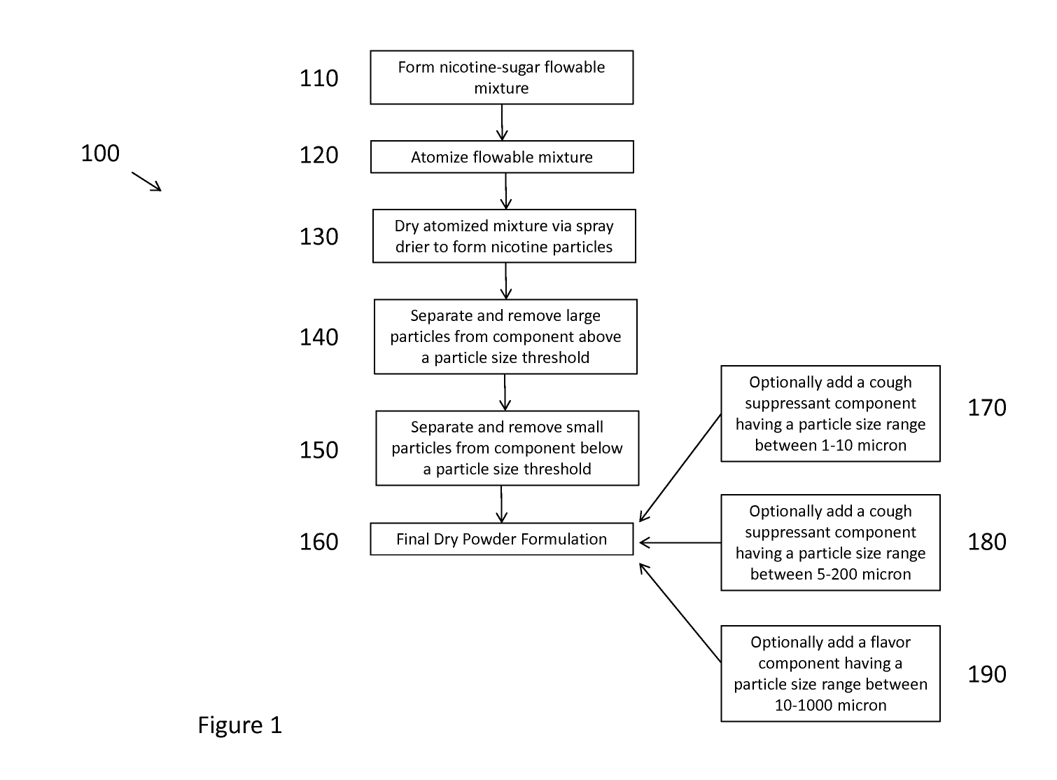

As shown in FIG. 1, the present invention includes a process or method 100 of producing any one of the formulations described herein. For example, in step 110, nicotine is admixed with a carrier, such as a sugar, for example lactose, to form a flowable mixture. At step 120, the mixture is atomized. At step 130, the mixture is dried, such as via a spray drier. Alternatively, the process may optionally be performed via fluid bed drying, wherein nicotine can instead be spray dried onto the lactose. At step 140, the resulting nicotine particles are filtered, such as with a sieve, to remove any particles larger than a threshold size value. At step 150, the resulting nicotine particles are filtered again to remove any particles smaller than a threshold size value, resulting in the final dry powder formulation. In some embodiments, only one filtering step is needed. In other embodiments, two or more filtering steps are needed. Optionally at step 170, a cough suppressant component may be added to final formulation 160. Step 170 may contain any number of processing steps needed to obtain the desired particle size (e.g., 1-10 micron) for the cough suppressant component being added. Optionally at step 180, a cough suppressant component may be added to final formulation 160. Step 180 may contain any number of processing steps needed to obtain the desired particle size (e.g., 10-200 micron) for the cough suppressant component being added. Optionally at step 190, a flavor component may be added to final formulation 160. Step 190 may contain any number of processing steps needed to obtain the desired particle size (e.g., 10-1000 micron) for the flavor component being added.

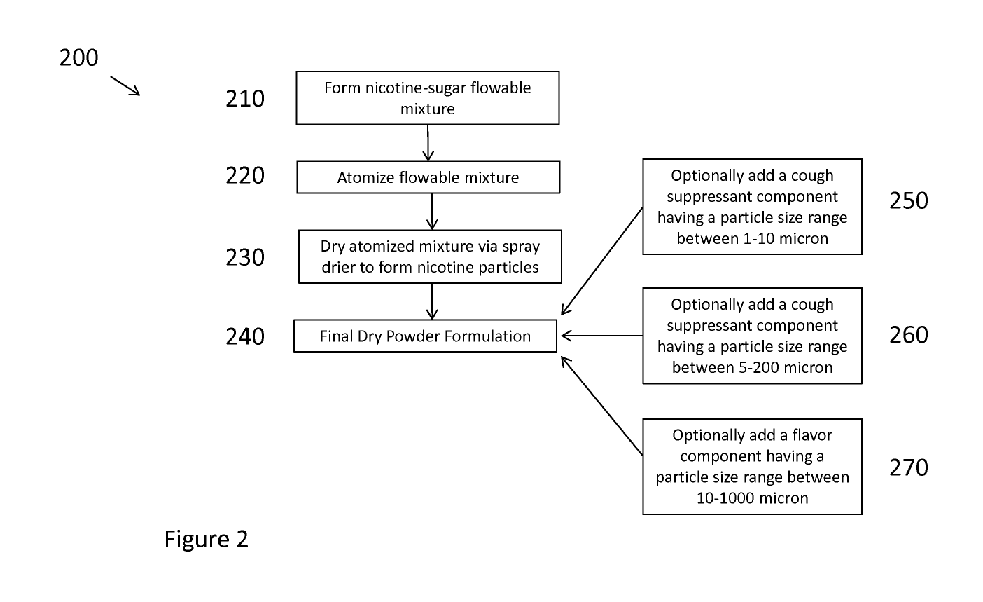

Alternatively, the formulations are produced without a filtering step, and instead the particles are generated within the desired size range. By controlling the size of the particles generated to substantially those desired, filtration steps may not be necessary. For example, as shown in FIG. 2, the present invention includes a process or method 200 of producing any one of the formulations described herein. For example, in step 210, nicotine is admixed with a carrier, such as a sugar, to form a flowable mixture. At step 220, the mixture is atomized. At step 230, the mixture is dried, such as via a spray drier, such that the resultant particles formed are substantially within the desired size range (in dry powder form). Optionally at step 250, a cough suppressant component may be added to final formulation 240. Step 250 may contain any number of processing steps needed to obtain the desired particle size (e.g., 1-10 micron) for the cough suppressant component being added. Optionally at step 260, a cough suppressant component may be added to final formulation 240. Step 260 may contain any number of processing steps needed to obtain the desired particle size (e.g., 10-200 micron) for the cough suppressant component being added. Optionally at step 270, a flavor component may be added to final formulation 240. Step 270 may contain any number of processing steps needed to obtain the desired particle size (e.g., 10-1000 micron) for the flavor component being added.

In one embodiment, the nicotine-based component may include nicotine and a pharmaceutical grade sugar prepared as solid discrete flowable particles, which may be entrained in the air inhaled by a subject so as to travel to the alveoli and smaller airways of the lungs. Further, the dried nicotine-sugar particles may be filtered, such as via one or more sieving steps, to isolate and segregate the desired particle sizes from those particles being removed.

In one embodiment, initial particles of the nicotine-based component may be produced via the methods as described in U.S. Patent Application Publication No. 20120042886, which is incorporated by reference herein in its entirety. For example, in a first step, nicotine and a pharmaceutical grade sugar, such as lactose, can be mixed with a liquid carrier so as to form a flowable mixture.

As contemplated herein, the sugar is an inhalable sugar, and is generally soluble in a liquid carrier, such as water. Without limitation, examples of suitable sugars are lactose, sucrose, raffinose, trehalose, fructose, dextrose, glucose, maltose, mannitol, or combinations thereof. In a preferred embodiment, the sugar may be alpha monohydrate lactose. The sugar may be a natural or a synthetic sugar, and may include any analogs or derivatives of sugars. It should be appreciated that any form of sugar approved as an excipient may be used as a carrier in the production of the nicotine-based component. While not required, the sugar is preferably of a pharmaceutical grade as would be understood by those skilled in the art. Preferably, the pharmaceutical grade sugar used to create the flowable mixture is a non-spheronized sugar. Surprisingly, the form or shape of the pharmaceutical grade sugar that is combined with nicotine when forming the flowable mixture affects the final shape of the nicotine-based particles produced. In particular, when non-spheronized sugar is substantially solubilized and mixed with nicotine, substantially spherical nicotine-sugar particles are formed when spray dried. However, if a spheronized sugar is used to form the flowable mixture, then the resultant spray dried product tends to form as string shaped particles, instead of the desired spherical particles. Accordingly, the pharmaceutical grade sugar may be prepared in a non-spheronized form prior to admixture with nicotine. For example, the pharmaceutical grade sugar may be first prepared in a non-spheronized form by freeze drying, milling, micronizing or the like. In certain embodiments, the pharmaceutical grade sugar may be subjected to milling, bashing, grinding, crushing, cutting, sieving or other physical degradation process as understood by those skilled in the art, which ultimately reduces the particle size of the sugar and results in a non-spheronized sugar.

As contemplated herein, any form of nicotine may be used for admixture with the sugar to form the nicotine-based component. Preferably, a form of nicotine which is soluble in or miscible with the liquid carrier, is used. For example, the nicotine may be nicotine base, which, at room temperature, is a liquid that is miscible in water. Alternatively, nicotine base may be used in an oil formulation. In one embodiment, the nicotine is a salt, which, at room temperature, is a solid. The nicotine may further be a pharmacologically active analog or derivative of nicotine or substance that mimics the effect of nicotine, either alone or in combination with other active substances. If the nicotine is a base, then it may be added to the liquid carrier (such as water) and mixed to produce a generally homogeneous liquid mixture.

Accordingly, in one embodiment, nicotine is present in the formulation as a free base. In another embodiment, the formulation may comprise a nicotine salt. In one such embodiment, the nicotine salt is nicotine hydrogen tartrate. In other embodiments, the nicotine salt can be prepared from any suitably non-toxic acid, including inorganic acids, organic acids, solvates, hydrates, or clathrates thereof. Non-limiting examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

In various embodiments, the formulation can further comprise any pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Each material must be “acceptable” in the sense of being compatible with the other ingredients of the formulation, including nicotine, and not injurious to the subject. Some materials that may useful in the formulation of the present invention include pharmaceutically acceptable carriers, for example sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar;

buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

Other pharmaceutically acceptable materials that can be useful in the formulation include any type of coating, antibacterial and antifungal agents, absorption delaying agents, and the like that are compatible with the activity of nicotine or any other compound useful within the invention, and are physiologically acceptable to the subject. Supplementary active compounds, including pharmaceutically acceptable salts of those compounds, may also be incorporated into the compositions. Other additional ingredients that may be included in the compositions used in the practice of the invention are known in the art and described, for example in Remington's Pharmaceutical Sciences (Genaro, Ed., Mack Publishing Co., 1985, Easton, Pa.), which is incorporated herein by reference.

As contemplated herein, any liquid carrier may be used in the process of producing the nicotine-based component. Preferably, the liquid carrier is one in which both the pharmaceutical grade sugar and the nicotine base are soluble. For example, in one embodiment, the liquid carrier is water. While water is the preferred liquid carrier, other liquids in combination with or in place of water may be used. For example, the liquid carrier may comprise a mixture of an alcohol and water to form an azeotropic liquid carrier. If an alcohol is used, the alcohol is preferably a primary alcohol. In one embodiment, the alcohol is preferably a lower alkyl alcohol (i.e. C₁ to C₅), such as ethanol. In such embodiments, any ratio of water to alcohol may be used, and may be determined when balancing the solubility of the mixture components with the desired drying rate of the final mixture. In some embodiments, the ratio of alcohol to water in the liquid carrier may be from about 1:1 to 1:10, preferably from about 1:2 to 1:8 and more preferably from about 1:5 to 1:7 parts by weight. Accordingly, the liquid carrier may be any liquid or liquids with which nicotine may be admixed with sugar to form a flowable mixture which is preferably of a generally uniform composition.

It should be appreciated that there are no limitations to the ratio of nicotine to sugar used, and the actual ratio used will be based on the concentration of nicotine desired in the nicotine based component particles. Accordingly, in one embodiment the ratio of sugar to nicotine in the flowable mixture may vary from about 1:100 to about 100:1, or from about 3:7 to about 3:2 or alternatively, from about 4:6 parts by weight. Further, the concentration of sugar in the flowable mixture may vary from about 1 to about 10 w/v (g/100 ml), from about 2 to about 5 w/v (g/100 ml) or from about 3% w/v (g/100 ml). In a preferred embodiment, the concentration of nicotine is between about 5-10%.

As mentioned previously, the nicotine-sugar flowable mixture is dried, such as via a spray drier, to produce composite particles of nicotine-sugar that are suitable for delivery to the alveoli and lower airways of a subject. It should be appreciated that there is no limitation to the method of drying the flowable mixture.

While a preferred method utilizes a spray drier, other drying techniques capable of producing appropriately sized particles may be used, such as fluidized bed drying. In one embodiment, the mixture is finely divided via passage through an orifice upon on entry to a spray dryer. In another embodiment, the flowable mixture may be passed through an atomizer, such as a rotary atomizer, to feed the flowable liquid into a spray dryer.

Further still, any rate of drying may be used (e.g., slow or rapid rate drying), provided such rate of drying results in the formation of dry particles of the desired size range. Prior to the segregation of the desired particle size of the nicotine-based component, the resultant particles formed via the spray drier may have a particle size from about 0.1 to about 5 micron.

While additional segregation/filtering of selected particle sizes may be performed subsequent to the formation of the nicotine based component dry particles, the operating conditions of the spray dryer may be adjusted so to produce particles which are sized so as to be able to travel to the alveoli and smaller airways of the lungs. For example, a rotary atomizer may be operated at a liquid feed rate from about 2 to about 20 ml/min, or from 2 to about 10 ml/min, or from about 2 to about 5 ml/min. Further, the rotary atomizer may be operated from about 10,000 to about 30,000 rpm, from about 15,000 to about 25,000 rpm, or from about 20,000 to about 25,000 rpm. It should be appreciated that particles of various sizes may be obtained by spray drying, and particles having the desired particle size may be more specifically selected when filtered, such as via one or more sieving steps, as described elsewhere herein. The spray dryer may be operated at temperatures sufficiently high to cause the liquid carrier to rapidly evolve without raising the temperature of the sugar and nicotine within the mixture to a point at which these compounds begin to degrade. Accordingly, the spray dryer may be operated with an inlet temperature from about 120 to about 170° C. and an outlet temperature from about 70 to about 100° C.

It should be appreciated that the nicotine-based component particles may be spherical or of any other shape desired. In one embodiment, by evolving the liquid carrier sufficiently rapidly during the spray drying process, the particles may be produced with an uneven or a “dimpled” surface. In such embodiments, the uneven surface may produce a relative turbulence as the particles travel through the air, thus providing the particles with aerodynamic lift. In such embodiments, particles having such shape may be more readily entrained, and to remain entrained, in the air inhaled by a subject, thereby improving the ability of the nicotine-based component particles to travel to the alveoli and smaller airways.

As mentioned previously, the present invention includes formulations having components characterized by particular particle size ranges. For example, the formulations of the present invention can include nicotine-based particles sized substantially between 1-10 microns, and preferably between 2-5 microns. In other embodiments, the formulations can optionally include a cough suppressant component (such as menthol) having particles in the size range of 1-100 microns. In other embodiments, the formulations can optionally include a second cough suppressant component having particles in the size range of 10-200 microns. In further embodiments, the formulations can include a flavor component (such as menthol) having particles in the size range of 10-1000 microns.

As contemplated herein, the particles of the present invention can be produced in relatively narrow size ranges via the use of at least one sieving step. In such an embodiment, the sieving step includes using a sieve corresponding to the minimum or maximum of the desired particle size range to eliminate particles from the mixture that are smaller or bigger than the desired range. For example, to obtain nicotine particles in the range of about 1-5 microns, a mixture of nicotine particles produced using the spray drying process described herein can be provided. The mixture of nicotine particles will have a size distribution that is dependent on the spray dryer conditions used and/or the characteristics of the input mixture to the spray dryer. The mixture of nicotine particles can first be passed through a 5 micron sieve, wherein substantially all of the particles smaller than 5 microns pass through the sieve and are collected. The particles passing through the sieve can then transferred to a 1 micron sieve, wherein substantially all of the particles greater than 1 micron do not pass through the sieve. The particles greater than 1 micron can be collected from the sieve, wherein the collected particles will be substantially sized in the range of 1-5 microns. Accordingly, such a process can be used to narrow the range of any mixture of particles to any of the desired particle size ranges as described hereinthroughout.

In another embodiment, a mixture of particles can be provided that substantially meets either the minimum or maximum criteria of the desired particle size range. For example, if a nicotine particle size range of 2-3 microns is desired, a mixture of nicotine particles can be provided wherein substantially all of the particles are less than 3 microns. Such a mixture can be produced by modifying the spray dryer conditions, or by milling the spray dried material to result in a mixture of particles that are generally less than 3 microns. The mixture can then be transferred through a 2 micron sieve, wherein the particles not passing through the sieve are collected, and wherein the collected particles are substantially within the desired 2-3 micron range.

In one embodiment, a mixture of particles that substantially meets any particle size range criteria described herein can be provided via dry processes, for example by using dry process techniques such as milling, blending, and/or sieving. The dry process techniques can be used instead of or in addition to wet process techniques.

In one embodiment, the nicotine based component, cough suppressant component, flavor component, and/or any other type of component can be blended together to form a mixture having the desired particle size profile. Any method of blending particles can be used for the methods and formulations described herein. The blending can be conducted in one or more steps, in a continuous, batch, or semi-batch process. For example, if both a cough suppressant component and a flavor component are used, they can be blended together before, or at the same time as, being blended with the nicotine based component. The blending process may be performed using a variety of blenders. Representative examples of suitable blenders include V-blenders, slant-cone blenders, cube blenders, bin blenders, static continuous blenders, dynamic continuous blenders, orbital screw blenders, planetary blenders, Forberg blenders, horizontal double-arm blenders, horizontal high intensity mixers, vertical high intensity mixers, stirring vane mixers, twin cone mixers, drum mixers, and tumble blenders. The blender preferably is of a strict sanitary design required for pharmaceutical products.

Tumble blenders are often preferred for batch operation. In one embodiment, blending is accomplished by aseptically combining two or more components (which can include both dry components and small portions of liquid components) in a suitable container. One example of a tumble blender is the TURBULA™, distributed by Glen Mills Inc., Clifton, N.J., USA, and made by Willy A. Bachofen AG, Maschinenfabrik, Basel, Switzerland.

For continuous or semi-continuous operation, the blender optionally may be provided with a rotary feeder, screw conveyor, or other feeder mechanism for controlled introduction of one or more of the dry powder components into the blender.

In one embodiment, one or more milling steps can be used to fracture and/or deagglomerate the various component particles, to achieve a desired particle size and size distribution, or to enhance distribution of the particles within the blend. The one or more milling steps can be used before or after blending the various component particles together. In one embodiment, the process of milling two or more component particles can also be used to blend the particles, i.e., the milling and blending steps can be performed at the same time.

Any method of milling can be used to form the particles of the invention, as understood by one of ordinary skill in the art. A variety of milling processes and equipment known in the art may be used. Examples include hammer mills, ball mills, roller mills, disc grinders, jet milling and the like. Preferably, a dry milling process is used.

In addition, one or more sieving steps can be used either before or after the milling and/or blending steps to generate a mixture of component particles that meets the particles size criteria described herein. Non-limiting examples of sieving steps have been previously described herein.

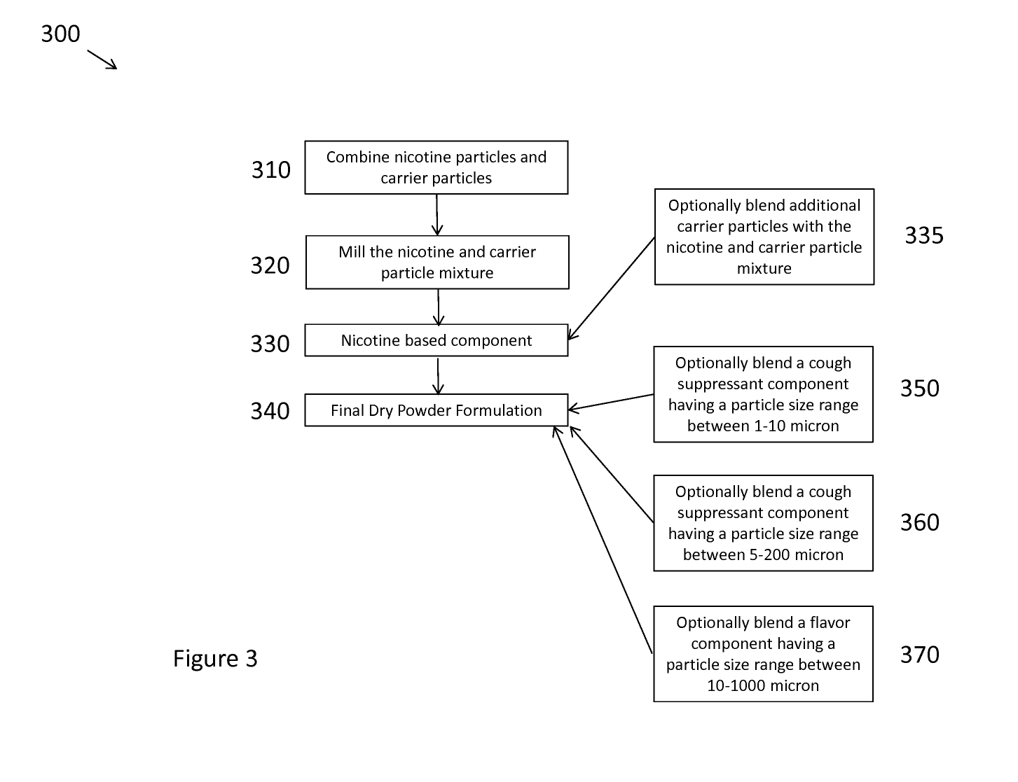

Referring now to FIG. 3, a diagram of a dry process or method 300 of producing any one of the formulations described herein is shown. For example, in step 310, nicotine particles are combined with a carrier, such as a sugar, preferably lactose. The nicotine particles can be any form of nicotine as described herein, for example nicotine tartrate. In one embodiment, the nicotine and carrier mixture can be combined via a spray drying process as previously described or via any other wet or dry process. In another embodiment, the nicotine and carrier mixture can be combined via dry blending. In yet another embodiment, the nicotine and carrier mixture are combined without being blended or mixed together. In one embodiment, the nicotine and carrier mixture in step 310 is about 1:1 nicotine:lactose. However, the ratio of nicotine:lactose is not limited to any specific ratio described herein.

At step 320, the nicotine and carrier mixture is milled to form the nicotine based component 330. In one embodiment, the milling of the nicotine and carrier mixture is used to blend the mixture to form a relatively uniform nicotine based component. In one embodiment, the average size of the nicotine particles are reduced to a greater degree than the average size of the carrier particles during milling step 320, i.e., the post-milling size of the nicotine particles and carrier particles is different. Optionally at step 335, additional carrier particles may added to the nicotine based component 330. The carrier particles added in step 335 can have the same composition and/or particles size as the carrier particles in step 310, or the carrier particles added in step 335 can have a different composition and/or particles size as the carrier particles in step 310. In one embodiment, the additional carrier particles added in step 335 can have a larger particle size than the carrier particles in the milled nicotine and carrier mixture. In one embodiment, the carrier particles added in step 335 are in the range of about 5-10 micron. In one embodiment, the nicotine based component 330 is about 1.5 to 7% nicotine particles, with the balance being carrier particles. For example, in one embodiment, the nicotine based component 330 is about 1.5 to 7% nicotine tartrate and about 93 to 98.5% lactose.

In one embodiment, nicotine based component 330 is the final dry powder formulation 340. In other embodiment, final dry powder formulation 340 can include other components. Optionally at step 350, a cough suppressant component may be added to final formulation 340. Step 350 may include any number of processing steps needed to obtain the desired particle size (e.g., 1-10 micron) for the cough suppressant component being added. Optionally at step 360, a cough suppressant component may be added to final formulation 340. Step 360 may include any number of processing steps needed to obtain the desired particle size (e.g., 10-200 micron) for the cough suppressant component being added. Optionally at step 370, a flavor component may be added to final formulation 340. Step 370 may include any number of processing steps needed to obtain the desired particle size (e.g., 10-1000 micron) for the flavor component being added.

In another embodiment, no carrier is added prior to milling, i.e., the milling step is performed only on nicotine particles. In one such embodiment, the nicotine particles alone can be used as the nicotine based component. In another such embodiment, carrier particles can be added to the milled nicotine particles to form the nicotine based component. In another such embodiment, the milled nicotine particles alone can be used as the final dry powder formulation. In yet another such embodiment, one or more cough suppressants and/or flavor components can be added to the milled nicotine particles to form the final dry powder formulation.

As would be understood by a person skilled in the art, the particle size ranges described herein are not absolute ranges. For example, a nicotine particle mixture of the present invention with a size range of 2-3 microns can contain a portion of particles that are smaller or larger than the 2-3 micron range. In one embodiment, the particle size value as presented for any particular component of the formulations of the present invention represents a D90 value, wherein 90% of the particles sizes of the mixture are less than the D90 value. In another embodiment, the particle size range represents a particles size distribution (PSD) wherein a percentage of the particles of the mixture lie within the listed range. For example, a nicotine particle size range of 2-3 microns can represent a mixture of nicotine particles having at least 50% of the particles in the range of 2-3 microns, but more preferably a higher percentage, such as, but not limited to: 60%, 70%, 80%, 90%, 95%, 97%, 98% or even 99%.

It is contemplated that the percentage of particles falling within the desired particle size range for any of the components of the formulation of the present invention can be dependent on the technique used to produce that component. For example, if the targeted size of the nicotine component is in the range of 2-5 micron, it is understood that greater than 90% of that component will fall within the desired range when using a spray drying production technique on a relatively small scale. However, using a relatively large scale spray drying production technique may only yield greater than 70% of the nicotine component within such a targeted range.

As mentioned previously, the formulation may optionally include a cough suppressant component, wherein the particles of the cough suppressant component are sized between about 5 and 10 micron. By selectively including menthol particles sized between 5-10 microns, these non-respirable menthol particles can reduce cough by soothing irritation in the subject's larger airways. In another example, the formulation of the present invention may optionally include a cough suppressant component having particles sized substantially between 10-200 microns. This cough suppressant component may reduce a cough caused by irritation of the oro-pharynx, the glottis vocal cords and other anatomic regions more proximal or closer to the mouth that contain receptors that can trigger cough or trigger other unwanted sensations. As contemplated herein, these larger particles do not enter the sub-glottic airways because of their momentum.

In one embodiment, the cough suppressant component of either the 5-10 or 10-200 micron ranges comprises menthol. Further, it should be appreciated that any other cough suppressant compounds may be used instead of or in addition to menthol, without limitation.

As contemplated herein, any form of menthol, such as a solid form of menthol can be used for processing into menthol particles useful within the present invention. Non-limiting examples of solid forms of menthol include powders, crystals, solidified distillate, flakes, and pressed articles. In one embodiment, menthol is in the form of crystals. Menthol can be processed into particles of a size ranging from about 5 μm to about 10 μm using any method known in the art. In some embodiments, menthol is admixed with further liquid or solid additives for processing. Particulate additives can furthermore also be used. In one embodiment, menthol is admixed with silicon dioxide. In another embodiment, menthol is admixed with a sugar, such as lactose. In some embodiments, the menthol is processed in a liquid carrier.

As contemplated herein, any liquid carrier may be used in the process of producing the menthol particles. In one embodiment, the liquid carrier is water. Preferably, the liquid carrier is one in which the menthol is soluble. Accordingly, the liquid carrier may be any liquid or liquids with which menthol, either alone or in combination with an additional component, forms a flowable mixture which is preferably of a generally uniform composition.

The menthol flowable mixture may be dried, such as via a spray drier, to produce composite particles of menthol, alone or in combination with an additional component, that are suitable for delivery to the alveoli and lower airways of a person. It should be appreciated that there is no limitation to the method of drying the flowable mixture. Examples of methods for drying the flowable mixture include, but are not limited to, spray drying, vacuum drying, and freeze drying. Further still, any rate of drying may be used (e.g., slow or rapid rate drying), provided such rate of drying results in the formation of dry particles of the desired size range.

As mentioned previously, the formulation may optionally include a flavor component, wherein the particles of the flavor component are sized between about 10 and 1000 micron. In one embodiment, the flavor component comprises menthol and may be produced as previously described herein. When other flavoring compounds are used, any known processing steps suitable for such compounds may be used to produce the flavoring component within the desired particle size range of 10-1000 micron.

In various embodiments, the relative weight percentage of each component in the formulation of the present invention can be varied to achieve different characteristics. Thus, as one skilled in the art would understand, the relative weight percentages of the components can be modified for various reasons, for example, but not limited to: optimizing the cough suppressant performance of the formulation; varying or improving the taste of the formulation; and adjusting the relative dose of nicotine. In certain embodiments, the formulation can be about 1-20% by weight flavor component, with a preferred weight of 1-5% flavor component. In certain embodiments, the formulation can be about 1-10% by weight cough suppressant, with a preferred weight of 1-2.5% cough suppressant. In various embodiments, the remaining portion of the formulation, aside from any flavor components, cough suppressant components, carriers, or other components, is the nicotine component. In one embodiment, the formulation can be approximately 100% nicotine component.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A dry powder nicotine formulation suitable for inhalation comprising nicotine particles, wherein the nicotine particles are substantially between about 1-10 micron in size.

2. The formulation of claim 1, wherein the nicotine particles are substantially between about 2-5 micron in size.

3. The formulation of claim 1, wherein less than about 10% of the nicotine particles are less than about 1 micron in size.

4. The formulation of claim 3, wherein less than about 10% of the nicotine particles are less than about 2 micron in size.

5. The formulation of claim 1, wherein at least about 90% of the nicotine particles are less than about 10 micron in size.

6. The formulation of claim 5, wherein at least about 90% of the nicotine particles are less than about 5 micron in size.

7. The formulation of claim 1, wherein less than about 10% of the nicotine particles are less than about 1 micron in size and wherein at least about 90% of the nicotine particles are less than about 10 micron in size.

8. The formulation of claim 2, wherein less than about 10% of the nicotine particles are less than about 2 micron in size and wherein at least about 90% of the nicotine particles are less than about 5 micron in size.

9. A dry powder nicotine formulation suitable for inhalation comprising:
a nicotine based component having particles substantially between about 1-10 micron in size; and
a cough suppressant component having particles substantially between about 5-10 micron in size.

10. The formulation of claim 9, wherein the cough suppressant component comprises menthol.

11. The formulation of claim 10, wherein the nicotine based component particles are substantially between about 2-5 micron in size and the cough suppressant component particles are substantially between about 5-8 micron in size.

12. The formulation of claim 9, further comprising a cough suppressant component having particles substantially between about 10-200 micron in size.

13. The formulation of claim 12, wherein the cough suppressant component having particles substantially between about 10-200 micron in size comprises menthol.

14. The formulation of claim 9, further comprising a flavor component having particles substantially between about 10-1000 micron in size.

15. The formulation of claim 14, wherein the flavor component comprises menthol.

16. A method of producing a dry powder nicotine formulation suitable for inhalation, comprising the steps of:
preparing a flowable mixture comprising nicotine and a sugar in a liquid carrier; and
spray drying the flowable mixture to produce dry powder particles comprising nicotine and sugar, wherein the dry powder particles are substantially in the range of about 1-10 micron in size.

17. The method of claim 16, wherein the sugar is lactose.

18. The method of claim 17, wherein the lactose is non-spheronized.

19. The method of claim 16, wherein the liquid carrier is water.

20. The method of claim 16, wherein the liquid carrier comprises water and alcohol.

21. A method of producing a dry powder nicotine formulation suitable for inhalation, comprising the steps of:
preparing a mixture comprising nicotine particles and carrier particles; and
milling the mixture to produce a dry powder nicotine formulation having particles substantially in the range of about 1-10 micron in size.

22. The method of claim 21, wherein the nicotine particles comprise nicotine tartrate.

23. The method of claim 21, wherein the carrier particles comprise lactose.

24. The method of claim 21, wherein the dry powder nicotine formulation is about 1.5 to 7% nicotine.

25. The method of claim 21, further comprising adding additional carrier particles after milling.

26. The method of claim 21, further comprising adding a cough suppressant component having a particle size range between 5-10 micron after milling.

27. The method of claim 21, further comprising adding a cough suppressant component having a particle size range between 5-200 micron after milling.

28. The method of claim 21, further comprising adding a flavor component having a particle size range between 10-1000 micron after milling.
